(19) United States
(12) Patent Application Publication
(10) Pub. No.: US 2015/0075532 A1
(43) Pub. Date: Mar. 19, 2015

(54) Disposable Mask Made From Bioplastic Resins

(71) Applicant: Shantu Patel, Carlsbad, CA (US)

(72) Inventor: Shantu Patel, Carlsbad, CA (US)

(73) Assignee: INNOVATIVE BOTTLES, LLC, Carlsbad, CA (US)

(21) Appl. No.: 14/553,479

(22) Filed: Nov. 25, 2014

Related U.S. Application Data
(63) Continuation-in-part of application No. 14/483,155, filed on Sep. 11, 2014.
Application No. 14/483,155 is a continuation-in-part of application No. 13/590,377, filed on Aug. 21, 2012, now Pat. No. 8,852,157.
Application No. 13/590,377 is a continuation-in-part of application No. 14/515,468, filed on Oct. 15, 2014.

(51) Int. Cl. A61M 16/06; A61M 16/10; A61L 31/04; A61L 31/14; A61L 31/06

(52) U.S. Cl. CPC A61M 16/0622; A61L 31/14; A61L 31/141; A61L 31/06; A61L 31/042; A61L 31/048; A61M 16/0694; A61M 16/1005; A61M 16/104; A61M 2016/103
USPC 128/206.24

(57) ABSTRACT

A disposable mask made from bioplastic resins includes a biodegradable resin selected from the group consisting of polylactic acid (PLA), cellulose based PH, polycaprolate (PCL), polybutyleneadipatetetephathalate (PBT), polyhydroxyalkanoate (PHA), green polyethylene (GPE), green polyethylene terephthalate (GPET), Poly3-hydoxybutrate-3-hydroxyhexxanate (PHBH), poly-D-lactide (PDLA), and poly-L-lactide (PLLA); a plasticizer intermixed with the resin to provide a generally homogenous bioplastic; and a medical mask that includes a shell substantially made of the bioplastic and a biodegradable cushion attached to an edge of the shell.

### RELATED APPLICATIONS

This application claims the benefit of the filing date of U.S. patent application Ser. No. 13/590,377, filed Aug. 21, 2012, which is incorporated herein by reference in its entirety; U.S. patent application Ser. No. 14/483,155, filed Sep. 11, 2014, which is incorporated herein by reference in its entirety; and U.S. patent application Ser. No. 14/515,468, filed Oct. 15, 2014, which is incorporated herein by reference in its entirety.

### BACKGROUND OF THE INVENTION

The present invention generally relates to disposable, biodegradable items and more specifically to disposable masks and tubing made from bioplastic resins.

Environment and sustainability have become increasingly important factors in the design and specification of medical items and their safe disposal after use. Due to higher social responsibility and environmental concerns, corporations are being driven to produce more sustainable and environmentally safe products through government regulations, by institutional investors, and through consumer demand.

Bioplastic resins include Polylactic acid (PLA), cellulose based PH, polybutylene adipate terephthalate (PBT) and polycaprolate (PCL), from corn and cellulose; green polyethylene, (GPE) and green polyethylene terephthalate (GPET also known as GPETE) from sugarcane; and Poly3-hydoxybutrate-3-hydroxyhexxanate (PHBH) from a fermentation process using glucose and propionic acid as the carbon source for Alcaligenes eutrophus. PHA polyhydroxyalkanoate) is derived by plant fermentation. Poly L lactide (PLLA) and poly D lactide (PDLA) are forms or homo-polymers of PLA. PLA, PDLA, and PLLA are especially compostable and can be degraded to make eco-friendly compost or humus. Bioplastic resins include PLA, PHA, PCL, PH, PBT, GPE, GPET, PHBH, PDLA, and PLLA.

Bioplastic resins may have advantages over plastic and glass. Bioplastic has a smaller carbon footprint than plastic or glass, and also uses less energy to form an article like a biodegradable medical device. Bioplastics are biodegradable in an industrial composting unit. Bioplastic resins are from a plant source, and when plants are grown they absorb carbon dioxide, thus decreasing carbon dioxide in the atmosphere. These advantages, namely small carbon foot prints, absorbing carbon dioxide and using less energy, are up-stream advantages and the biodegradable and compostable advantages are downstream advantages. Plastic and glass disposable items have a higher carbon footprint than items made of other materials. Plastic and glass items enter the waste stream when they are disposed of, and may need incineration a process that causes release of hydrocarbons and toxins into the atmosphere and creates fly ash that ends up in landfills.

Items made from bioplastic resin such as PLA, PHA, PH, PCL PCH are all biodegradable. Compostable items may be sterilized and then shredded and composted. PLA derived polymers namely PLLA and PDLA offer higher heat distortion properties can also be used. This will allows them to be diverted from land field. Some bio resins, such as GPE and GPET, may be made from plant sources even thought they might not be fully biodegradable or compostable.

The use of face masks to apply inhalation agents (gases) is appropriate for anesthesia and in supporting ventilation for medical treatment of patients needing ventilation support or who suffer from sleep apnea. These same masks and tubing can also be used to treat patients with respiratory distress from respiratory illness like asthma and chronic obstructive pulmonary disease, having an exacerbation and need a breathing treatment, to deliver aerosolized inhalants to the lungs. For the administration of general anesthesia (GA) it is common to ventilate a patient with oxygen during the pre-intubation stage (induction) using a mask coupled to a suitable supply, using flexible breathing tubing. Sometimes it is necessary to continue to use mask ventilation during surgery when a patient cannot be intubated with an endotracheal tube, or during procedures of relatively brief duration. On other occasions, it may be desirable to provide supplemental anesthesia using various anesthesia inhalation agents in the form of a gas selected, for example, from the group consisting of desflurane, sevoflurane, isoflurane, or so nitrous oxide, or combinations thereof. Patients with sleep apnea may need positive ventilation support each night or during sleep, on a temporary basis. Oxygen or the inhalation gases selected by the anesthesiologist typically are applied to a patient using an inhalation face mask connected to a flexible tube or tubes which in turn is connected to a suitable gas supply. The fresh gas forces expired carbon dioxide (CO2) out of the mask to provide a breathing circuit. Generally, once a patient is ventilated and then intubated with an endotracheal tube or the like, the mask is set aside until the end of the procedure when the endotracheal tube is removed and the patient briefly is ventilated with the mask.

It would be desirable to provide bioplastic or compostable devices such as medical face masks.

### SUMMARY OF THE INVENTION

In one aspect of the present invention, a disposable device includes a biodegradable resin selected from the group consisting of polylactic acid (PLA), cellulose based PH, polycaprolate (PCL), polybutyleneadipatetetephathalate (PBT), polyhydroxyalkanoate (PHA), green polyethylene (GPE), green polyethylene terephthalate (GPET), Poly3-hydoxybutrate-3-hydroxyhexxanate (PHBH), poly-D-lactide (PDLA), and poly-L-lactide (PLLA); a plasticizer intermixed with the resin to provide a generally homogenous bioplastic; and a medical mask that includes a shell substantially made of the bioplastic and a biodegradable cushion attached to an edge of the shell.

In another aspect of the present invention, a medical mask includes a biodegradable resin selected from the group consisting of polylactic acid (PLA), cellulose based PH, polycaprolate (PCL), polybutyleneadipatetetephathalate (PBT), polyhydroxyalkanoate (PHA), green polyethylene (GPE), green polyethylene terephthalate (GPET), Poly3-hydoxybutrate-3-hydroxyhexxanate (PHBH), poly-D-lactide (PDLA), and poly-L-lactide (PLLA); a plasticizer intermixed with the resin to provide a generally homogenous bioplastic; a shell substantially made of the bioplastic, having an interior cavity and an edge, the shell having a nasal portion with a first width, a mouth portion with a second width greater than the first width, and a chin portion with a third width greater than the second width; an input port that provides gas to the interior cavity of the shell; an output port that expels gas from the interior cavity of the shell; and a biodegradable cushion attached to the edge of the shell.

### BRIEF DESCRIPTION OF THE DRAWINGS

FIG. 1 is a perspective view of an embodiment of a mask and tubing assembly according to the present invention;

FIG. 2 is a view of an embodiment of a disposable anesthesia mask according to the present invention, in position on a patient's face;

FIG. 3 is a top plan view of the mask of FIG. 2;

FIG. 3A is a top plan view of the mask of FIG. 2 depicting zones;

FIG. 4 is a side elevation view of the face mask of FIG. 2;

FIG. 5 is a cross-sectional view of a mask with pneumatic cushion according to the present invention;

FIG. 5A is an enlarged view of a portion of the mask of FIG. 5;

FIG. 6 is an exploded view of the mask of FIG. 5;

FIG. 7 is a fragmentary perspective view of a portion of the mask of FIG. 5;

FIG. 8 is a bottom plan view of the mask of FIG. 5; and

FIG. 9 is a schematic diagram showing a mask and tubing assembly with a breathing circuit and a CO2 monitor according to the present invention.

### DETAILED DESCRIPTION

The preferred embodiment and other embodiments, which can be used in industry and include the best mode now known of carrying out the invention, are hereby described in detail with reference to the drawings. Further embodiments, features and advantages will become apparent from the ensuing description, or may be learned without undue experimentation. The figures are not necessarily drawn to scale, except where otherwise indicated. The following description of embodiments, even if phrased in terms of “the invention” or what the embodiment “is,” is not to be taken in a limiting sense, but describes the manner and process of making and using the invention. The coverage of this patent will be described in the claims. The order in which steps are listed in the claims does not necessarily indicate that the steps must be performed in that order.

Disposable plastic articles used in medical offices, hospitals and healthcare industries may be made from sustainable, environmentally friendly bioplastic resins and may be safely disposed without further environmental impact. Embodiments of disposable articles may be made from bioplastic resin, which include PLA (including PLLA and PDLA), PHA, PCH, PCL, PH, PBT, GPE, GPET, and PHBH. Embodiments of disposable articles may include a plasticizer intermixed with the resin to provide a generally homogenous bioplastic. Embodiments may include a disposable mask and tubing.

An embodiment of the present invention may include a medical mask such as a disposable anesthesia face mask, ventilation mask, sleep apnea mask or respiratory mask (a “medical mask”). The mask may include a cup-like shell or receptacle member terminating at a peripheral edge with a circumferential flange, and an annular donut-shaped hollow cushion or seal member affixed or otherwise permanently attached to the flange. The seal member may be inflatable.

The shell or shell member may preferably be transparent to permit viewing therethrough. The shell member may be shaped suitably to define a nose reception portion, a mouth reception portion, and a chin reception portion. The shell member may include a first passageway extending from the shell member from in a direction opposite to the flange for coupling to a breathing circuit, and a second passageway that may extend in a direction opposite to the flange for output. Embodiments of an anesthesia or ventilation mask may be connect the output passageway to a flexible tube which, in turn, is adapted to be connected to a CO2 monitoring machine or capnograph.

The face mask nasal portion may have a first width, the face mask mouth portion may have a second width greater than the first width, and the face mask chin portion may have a third width greater than the second width, giving the face mask shell a characteristic pear-shaped configuration. The nasal, mouth and chin portions may be continuous and form the cup-like extent of the shell or receptacle component.

Embodiments may include a headband. The mask may have lateral projections on the flange or two sides of the shell for accommodating a suitable elastic head strap that may adjustably be coupled to the projections to thereby retaining the strap to the mask. The tension in the elastic headband when attached may provide a stable suspension system securely maintaining the mask assembly in place before or during surgery and enabling anesthesia personnel to attend to other tasks using both hands free. This also applies if the mask and band are used for sleep apnea or during a breathing treatment.

Embodiments of a mask may be adapted to be fitted to the patient's face with the chin portion of the face being received in a cavity near the bottom of the mask. T-shaped posts that hold the headband may be located on the flange member upwardly near the nose portion of the mask assembly. The lateral projections hold the strap to form a loop that holds the mask in place on the user's face.

An embodiment of a face mask may be made from bio resins for environmental advantages, using a bioresin such as PLA (including PLLA and PDLA), PHA, PBT, PH, PCH, PH, GPE , GPET, or PHBH. A plasticizer intermixed with the resin to provide a generally homogenous bioplastic, and the device may be substantially made from the bioplastic. These polymers offer environmental advantages and can be in any combinations. For example, the mask can be made from PLA and the cushion can be from PCL GPE, or GPET.

Embodiments of masks may include an anesthesia mask, ventilation mask, sleep apnea mask, or a respiratory therapy mask. Embodiments of masks may be disposable, made of transparent material, may have a strap or straps to hold the mask in place when desired. Embodiments may be of sufficient size to cover the patient's nose, mouth and chin. Embodiments may have a pneumatic sealing cushion, to promote patient comfort and to prevent leakage of air or gases once the mask is applied. Embodiments may include gas input ports or output ports.

As depicted in FIG. 1, an embodiment of a mask assembly 10 may include facemask 11 and tubing 12. Facemask 11, tubing 12, or both may be made substantially of biodegradable resin mixed with a plasticizer, and may provide compostable material.

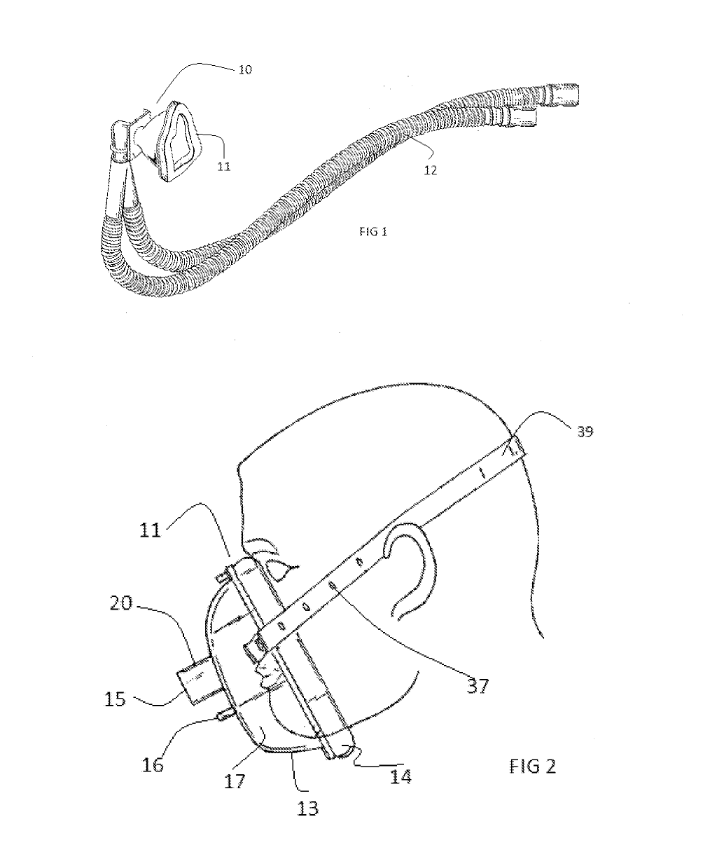

As depicted in FIG. 2, embodiments of face mask 11 may include a shell member 13 or other receptacle, and a cushion 14 that may be a pliable, or may be a flexible, pneumatically inflatable cushion. Embodiments of a shell member 13 may preferably be made of one-piece or unitary construction fabricated from bio resin. Shell member 13 may be transparent so that when face mask 11 is worn on the face of a patient, the portion of the patient's face covered or substantially worn by the mask is clearly visible at all times. Shell member 13 may have a first cylindrical hollow inlet member or port 15 that forms an input passageway 21, and a second cylindrical hollow outlet member or port 16 that forms an output passageway 22. Ports 15 and 16 may extend out from a top wall portion 17 that is opposite the user. In an embodiment, the location of ports 15 and 16 on the top wall portion 17 of shell member 13 may be such that when the mask 11 is suitably fitted to a patient's face, passageway 21 of inlet port 15 is located near the patient's nasal region, and passageway 22 of outlet port 16 is located near the mouth region of the patient. An input receptacle 20 may be adapted to attach tubes or tubing to the input port 15.

Embodiments of a mask 11 may be attached to the head of the patient using an elastic headband or strap member 39 selectively attachable to the mask shell 13 to adjust for different sizes. Strap member 39 may attached to mask 11 by way of a pair of protruding integral T-shaped posts 34, 35 extending from sides of the shell 13. Embodiments of a strap member 39 may preferably include a series of spaced holes 37 which are adapted to engage with the T-shaped posts 34, 35.

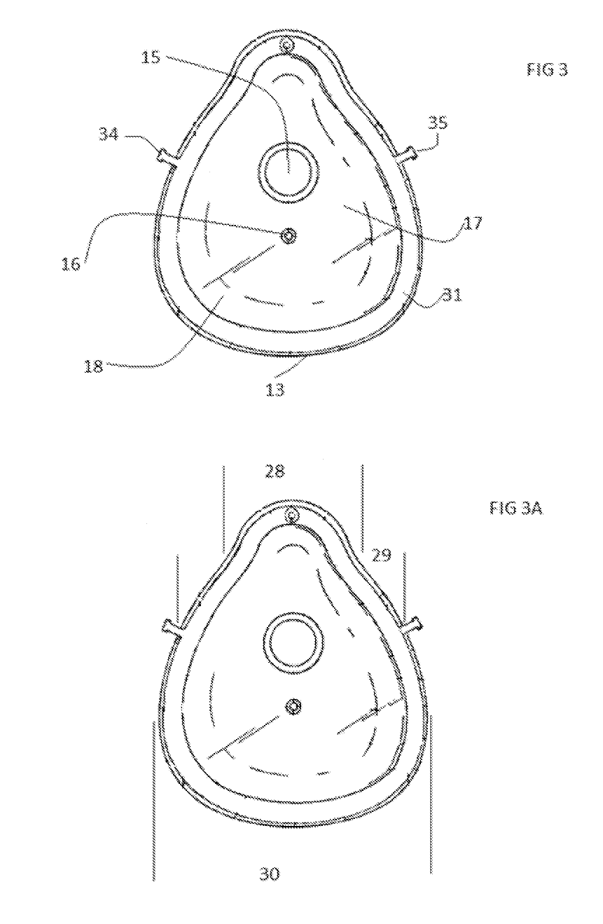

As depicted in the embodiment of FIG. 3, the outer or top wall portion of shell member 13 may extend slopingly into a continuous sidewall portion 18 along a peripheral extent of the shell member. Embodiments may include a pair of protruding integral T-shaped posts 34, 35 extending from sides of the shell 13. The T-shaped posts 34, 35 may fit into spaced holes in a headband or strap to hold the mask to the patient. Embodiments may include a gas input port 15, and an output port 16.

As depicted in the embodiment of FIG. 3A, a mask or shell may appear to be pear-shaped with a first transverse width or extent 28 at the front of the mask (top of the drawing) defining the nose reception region of the shell, a second or intermediate transverse width or extent 29 defining the mouth reception region of the shell, and a third transverse width or extent 30 at the rear defining the chin or jaw reception region of the shell, with the third extent 30 being greater than the intermediate extent 29 and the intermediate extent 29 being greater than the first transverse extent 28.

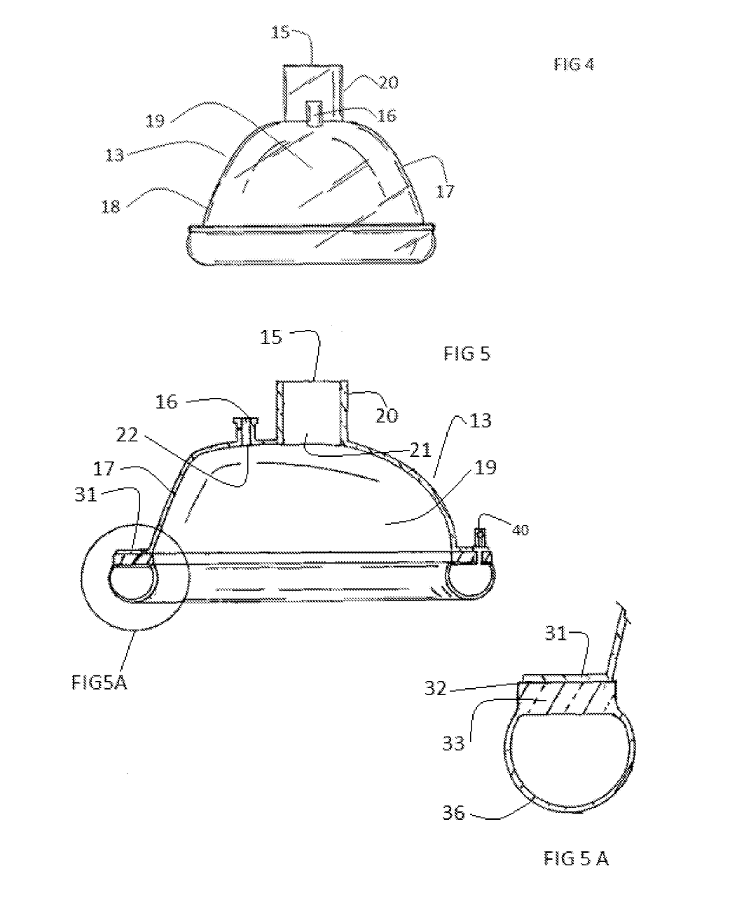

As depicted in the embodiment of FIG. 4, an outer or top wall portion 17 and continuous sidewall 18 of shell member 13 are concave defining a generally pear-shaped, cup-like receptacle having an interior cavity 19. Pear-shaped interior cavity 19 may be suitably shaped and sized to receive the nose portion, mouth portion and chin portion of a patient's face when the face mask assembly is sealingly applied to that patient's face. Embodiments may include a continuous sidewall portion 18 of shell member 13 which extends along the peripheral extent of the shell and terminates in a cushion 14.

As depicted in the embodiment of FIG. 5, a hollow inlet member or port 15 defines a first passageway 21 opening into interior cavity 19 and a second outlet member or port 16 defines a second passageway 22 opening into interior cavity 19. Port 15 may have an input receptacle 20. Embodiments may include a continuous sidewall portion or top wall portion 17 of shell member 13 which extends along the entire peripheral extent of the shell member 13 and terminates in a radially outwardly extending flange member 31. Embodiments may include a cushion inflation port 40 that allows a user to add or remove air from a pneumatic sealing cushion that attaches to the flange member 31.

FIG. 5A is a close-up of the circled area of FIG. 5. As depicted in the embodiment of FIG. 5A, the bottom of flange member 31 may be substantially flat so as to define a shell mounting surface 32. A pneumatic sealing cushion 36 may have a flat top that defines a pneumatic sealing cushion mounting surface 33. This allows embodiments to be made fabricated as by injection molding. A pneumatic sealing cushion 36 or cushion member preferably is formed with a somewhat thickened top wall portion defining a mounting foot, pad, or cushion mounting surface 33 which may be securely adhered to the mounting surface 32 of flange member 31 with an adhesive applied along the cushion mounting surface 33 surface of pad, the shell mounting surface 32, or both.

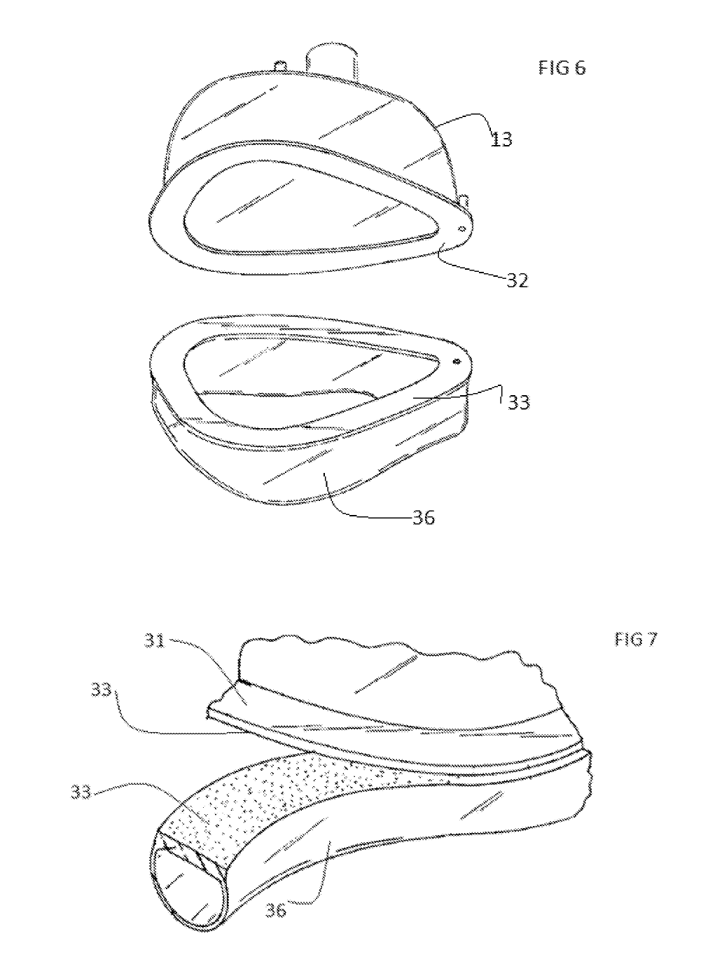

As depicted in the embodiment of FIG. 6, a shell member 13 with a flat lower surface may provide a shell mounting surface 32, and a pneumatic sealing cushion 36 with a flat upper surface may provide a cushion mounting surface 33 or other flexible annular sealing cushion component. The shell member 13 may have a cushion inflation port 40, that aligns with a cushion inflation conduit 41 in the pneumatic sealing cushion 36. The two parts mate and are fixed together, possibly with adhesive, so that air in the cushion 36 can be adjusted with the inflation port.

As depicted in the embodiment of FIG. 7, a flange member 31 on the shell has a lower shell mounting surface 32, which corresponds to and mates with a pneumatic sealing cushion 36 that has an upper cushion mounting surface 33.

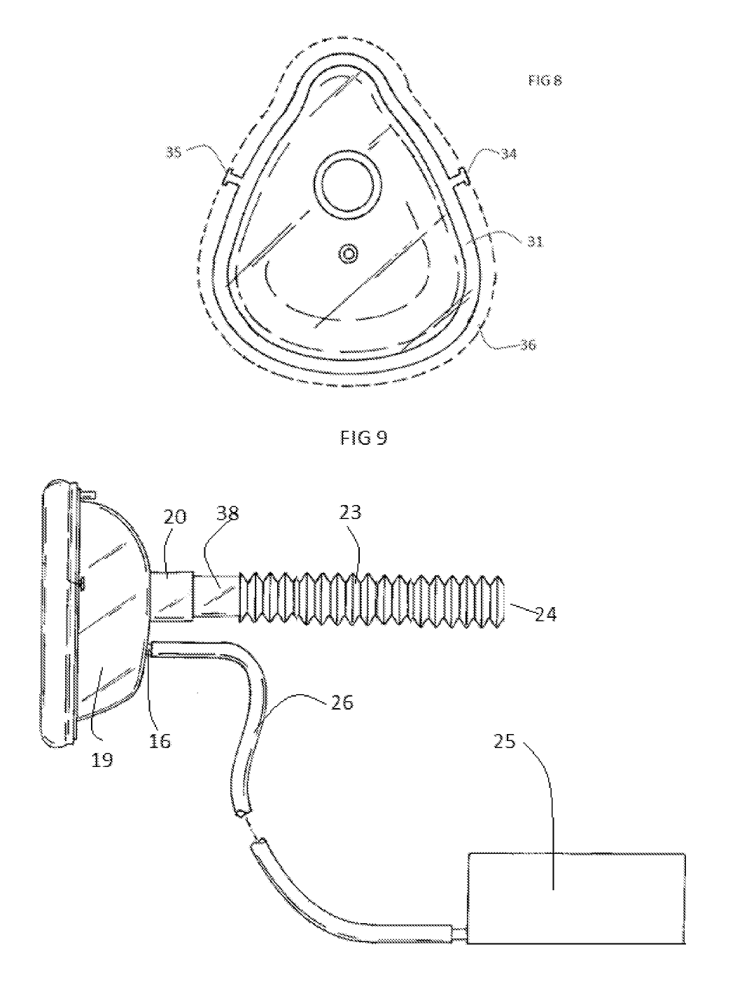

As depicted in the embodiment of FIG. 8, a pneumatic sealing cushion 36 includes a donut-shaped hollow annular member made of pliable flexible material, and can be inflatable. The pneumatic cushion member in FIG. 8 is shown in broken lines to avoid obfuscation. The sealing cushion 36 may be attached to a flange member 31, and may enable the shell member to be comfortably held to the face of the patient using an elastic headband or strap member selectively attachable to the mask shell via a pair of protruding 34, 35 integral T-shaped posts,

As depicted in the embodiment of FIG. 9, a mask assembly may have a mask made substantially of biodegradable resin or compostable material, attached to a breathing circuit and to a capnograph (CO2 monitor) through a flexible tube. In an embodiment, input receptacle 20 may be suitably sized to receive in slide so as to provide a snug, sealing engagement with a tube nipple 38 at the end of a flexible breathing tube 23. Flexible breathing tube 23 may attach to a breathing circuit 24 such that anesthesia gas or gases are adapted to pass from a source (not shown), through a branch of the breathing circuit 24, then through input receptacle 20, and into the interior cavity 19 of the facemask. The breathing circuit can be connected to a ventilator for ventilation support. For sleep, the breathing circuit may be connected to an apnea prevention machine or continuous positive airway pressure (CPAP) machine. The outside diameter of output port 16 may be suitably sized to snugly and sealingly fit on one end of a flexible CO2 tube 26 and the other end of the flexible CO2 tube 26 may be adapted connect to a conventional CO2 monitoring device 25 or capnograph. Embodiments of an outlet port 16 that provide CO2 may include a suitable screw-on type cap or closure member (not shown) when not being used.

I claim:

1. A disposable device comprising:
a biodegradable resin selected from the group consisting of polylactic acid (PLA), cellulose based PH, polycaprolate (PCL), polybutyleneadipatetetephathalate (PBT), polyhydroxyalkanoate (PHA), green polyethylene (GPE), green polyethylene terephthalate (GPET), Poly3-hydoxybutrate-3-hydroxyhexxanate (PHBH), poly-D-lactide (PDLA), and poly-L-lactide (PLLA);
a plasticizer intermixed with the resin to provide a generally homogenous bioplastic; and
a medical mask that includes a shell substantially made of the bioplastic and a biodegradable cushion attached to an edge of the shell.

2. The device of claim 1, wherein the shell includes a cup-like receptacle member terminating at a peripheral edge with a circumferential flange, and the cushion includes an annular donut-shaped hollow seal member affixed to the flange.

3. The device of claim 1, wherein the shell has an interior cavity, an input port that provides gas to the interior cavity, and an output port that expels gas from the interior cavity.

4. The device of claim 1, wherein the cushion is inflatable, further comprising a cushion inflation port that provides air in and out of the cushion.

5. The device of claim 1, wherein the medical mask has a nasal portion with a first width, a mouth portion with a second width greater than the first width, and a chin portion with a third width greater than the second width.

6. The device of claim 1, wherein the shell member is substantially made from the bioplastic, further comprising a cushion that includes a biodegradable resin.

7. The device of claim 1, further comprising a flexible input tubing that includes a biodegradable resin, adapted to attach to a gas input port of the mask.

8. The device of claim 1, further comprising a flexible output tubing that includes a biodegradable resin, adapted to attach to a gas output port of the mask.

9. The device of claim 1, further comprising:
lateral projections on two sides of the shell; and
a biodegradable strap having spaced holes shaped so that the lateral projections fit into the holes, thereby retaining the strap to the mask so as to form a loop that holds the mask in place on a user.

10. A medical mask comprising:
a biodegradable resin selected from the group consisting of polylactic acid (PLA), cellulose based PH, polycaprolate (PCL), polybutyleneadipatetetephathalate (PBT), polyhydroxyalkanoate (PHA), green polyethylene (GPE), green polyethylene terephthalate (GPET), Poly3-hydoxybutrate-3-hydroxyhexxanate (PHBH), poly-D-lactide (PDLA), and poly-L-lactide (PLLA);
a plasticizer intermixed with the resin to provide a generally homogenous bioplastic;
a shell substantially made of the bioplastic, having an interior cavity and an edge, the shell having a nasal portion with a first width, a mouth portion with a second width greater than the first width, and a chin portion with a third width greater than the second width;
an input port that provides gas to the interior cavity of the shell;
an output port that expels gas from the interior cavity of the shell; and
a biodegradable cushion attached to the edge of the shell.

11. The device of claim 10, wherein the biodegradable resin is PLA.

12. The device of claim 10, wherein the biodegradable resin is PH.

13. The device of claim 10, wherein the biodegradable resin is PCL.

14. The device of claim 10, wherein the biodegradable resin is PBT.

15. The device of claim 10, wherein the biodegradable resin is PHA.

16. The device of claim 10, wherein the biodegradable resin is GPE.

17. The device of claim 10, wherein the biodegradable resin is GPET.

18. The device of claim 10, wherein the biodegradable resin is PHBH.

19. The device of claim 10, wherein the biodegradable resin is PDLA.

20. The device of claim 10, wherein the biodegradable resin is PLLA.
